(19) United States
(12) Patent Application Publication
(10) Pub. No.: US 2026/0151250 A1
(43) Pub. Date: Jun. 4, 2026

(54) POSTURE CORRECTION DEVICE

(71) Applicant: Dongmei WEI, Tianjin (CN)

(72) Inventor: Dongmei WEI, Tianjin (CN)

(73) Assignee: PRANASH (TIANJIN) TECHNOLOGY DEVELOPMENT CO., LTD., Tianjin (CN)

(21) Appl. No.: 19/458,704

(22) Filed: Jan. 24, 2026

(30) Foreign Application Priority Data
Sep. 15, 2025 (CN) 202521978653.7

(51) Int. Cl. A61F 5/02

(52) U.S. Cl. CPC A61F 5/026; A61F 5/028

(57) ABSTRACT

A posture correction device, including: a main orthopedic band, an abdominal band, two shoulder straps, and two pulling straps. At least two main support rods are embedded in the main orthopedic band. The abdominal band symmetrically arranged at the main orthopedic band, which is configured to be fixed to a waist of a user through a free end of the abdominal band. Two shoulder straps symmetrically arranged at the main orthopedic band, each shoulder strap extends from under armpits of the user, pass over a respective shoulder, and reach a back. Each pulling straps is connected to one shoulder strap, and each shoulder strap is secured to the respective shoulder. By adjusting a connection length between the two pulling straps and the abdominal band, a traction system cooperates with a support system to achieve posture correction and stable support of shoulder-back and a waist-abdominal regions of the user.

### CROSS-REFERENCE TO RELATED APPLICATIONS

This application claims foreign priority of Chinese Patent Application No. 202521978653.7, filed on Sep. 15, 2025 in the China National Intellectual Property Administration, the disclosures of all of which are hereby incorporated by reference.

### TECHNICAL FIELD

The present disclosure relates to the technical field of ergonomic posture correction devices, and in particular, to a posture correction device.

### BACKGROUND

To actively correct body posture and alleviate discomfort, various types of posture correction devices have appeared on the market. These products can be broadly classified into shoulder and back correction belts mainly targeting the upper body, waist support belts focusing on the middle torso, and abdominal binders designed for tightening the abdomen, among others.

Most existing posture correction devices adopt a structure consisting of elastic materials combined with simple straps, which passively correct posture by applying backward or inward pulling forces on the shoulders or waist through physical restraint.

However, such designs exhibit significant limitations in practical use. Conventional shoulder straps lack scientific force direction guidance and limiting structures. After wearing, they are prone to slippage or cause localized excessive compression on the shoulders and underarms. They cannot precisely control scapular adduction and depression, and long-term use may lead to impaired blood circulation or induce compensatory muscle tension.

Moreover, most products rely solely on the tightening force of elastic bands, resulting in a single correction mode. They fail to systematically integrate and dynamically distribute the backward pulling force on the shoulder-back region, the horizontal encircling force on the waist-abdomen region, and the vertical supporting force in the lumbar region. Consequently, it is difficult to effectively and synergistically improve complex poor postures such as anterior pelvic tilt and thoracic kyphosis (hunchback). Overall, the correction effect is limited and wearing comfort is unsatisfactory.

In attempts to improve correction effectiveness and wearing experience, some improved designs incorporating rigid support strips or more segmented strap arrangements have emerged on the market. However, these designs typically merely superimpose additional functions without constructing an organic system in which various components cooperate in force bearing and are linked in adjustment.

Adding rigid supports may introduce new sensations of stiffness and restricted mobility, while complex multi-strap configurations make the wearing, removal, and adjustment processes cumbersome, making it difficult for users to quickly and accurately achieve overall fit according to their individual body type and correction needs. Furthermore, the lack of optimized design for the force transmission path causes dispersion of the corrective force or failure to maintain it persistently, thereby affecting the practical efficacy of the product and users'willingness to use it long-term.

In view of the above shortcomings, and particularly to overcome the contradiction existing in conventional posture correction devices that makes it difficult to simultaneously achieve systematic application of corrective forces, convenient adjustment operation, and good wearing comfort, the present disclosure provides a posture correction device featuring innovative structure, scientific support, and convenient personalized adjustment.

### SUMMARY

In order to overcome the deficiencies of the prior art, the present disclosure provides a posture correction device with innovative structure, safety, reliability and ease of manufacture.

To realize the above objective, the present disclosure provides a posture correction device, including: a main orthopedic band, wherein at least two main support rods are embedded in the main orthopedic band along a spinal direction, and a main support component is provided in a middle part of the main orthopedic band; an abdominal band symmetrically arranged at two lower lateral sides of the main orthopedic band, wherein the main orthopedic band is configured to be fixed to a waist of a user through a free end of the abdominal band; two shoulder straps symmetrically arranged at upper lateral sides of the main orthopedic band, each of the two shoulder straps is configured to extend from under left and right armpits of the user, pass over a respective shoulder, and reach a back of the user; two pulling straps, wherein each of the two pulling straps is fixedly connected to one of the two shoulder straps, and each of the two shoulder straps is secured to the respective shoulder; by adjusting a connection length between the two pulling straps and the abdominal band, a traction system constituted by the two shoulder straps and the two pulling straps cooperates with a support system constituted by the main orthopedic band to achieve posture correction and stable support of a shoulder-back region and a waist-abdominal region of the user.

Furthermore, the posture correction device further includes a waist compression strap fixedly connected to both sides of the main support component, and the main support component is connected with the abdominal band by a free end of the waist compression strap.

Furthermore, the posture correction device further includes auxiliary support components, wherein the auxiliary support components are symmetrically arranged on two sides of the main support component; by adjusting the connection length between the two pulling straps and the abdominal band and tightening the waist compression strap, the traction system constituted by the two shoulder straps and the two pulling straps, the support system constituted by the main orthopedic band, and an encircling system constituted by the abdominal band cooperate to achieve posture correction and stable support of the shoulder-back region and the waist-abdominal region.

Furthermore, an arc-shaped support portion adapted to a physiological curvature of a lumbar spine is embedded in the main support component, and an auxiliary support rod is provided inside each of the auxiliary support components.

Furthermore, a limiting portion is provided on the main orthopedic band, and each of the two pulling straps is passed through the limiting portion in an X-shaped crossing manner, so as to restrict a stretching direction of the two pulling straps.

Furthermore, two rib limiting straps are provided on the abdominal band, and each of the two pulling straps sequentially passes through the limiting portion and a respective rib limiting strap of the two rib limiting straps, and the respective rib limiting strap of the two rib limiting straps is finally configured to fix one of the two shoulder straps to the respective shoulder of the user by the free end of a respective pulling strap of the two pulling straps.

Furthermore, the limiting portion includes a first sliding groove and a second sliding groove crossing each other in an X shape, and the two pulling straps are slidably threaded along the first sliding groove and the second sliding groove, respectively.

Furthermore, the waist compression strap includes an upper compression strap and a lower compression strap, wherein the upper compression strap is fixedly connected to an upper lateral side of the main support component, and the lower compression strap is fixedly connected to a lower lateral side of the main support component.

Furthermore, after free ends of the upper compression strap and the lower compression strap are fixedly connected to the main support component, the main support component is connected with the abdominal band.

Furthermore, the abdominal band is a first hook-and-loop fastener, wherein a hook surface of the first hook-and-loop fastener is disposed on an inner side of the abdominal band, and a loop surface of the first hook-and-loop fastener is disposed on an outer side of the abdominal band.

Furthermore, each of the two pulling straps is a second hook-and-loop fastener, wherein a hook surface of the second hook-and-loop fastener is disposed on each of the two pulling straps.

Furthermore, the waist compression strap is a third hook-and-loop fastener, wherein a hook surface of the third hook-and-loop fastener is disposed on the waist compression strap.

Furthermore, the main orthopedic band is of an integrated back-waist structure.

Furthermore, the at least two main support rods are symmetrically arranged parallel to a spine centerline.

Furthermore, a shaping support rod is embedded in a connection portion between the abdominal band and the main orthopedic band.

Furthermore, when the posture correction device is in a worn state, the main support component and the arc-shaped support portion are positioned to fit a lumbar region of the user, the auxiliary support components and the auxiliary support rod are positioned to fit regions on both sides of the lumbar spine, and a hardness of the arc-shaped support portion is greater than a hardness of the auxiliary support rod.

Furthermore, a hardness of the arc-shaped support portion is greater than a hardness of the at least two main support rods, and the hardness of the at least two main support rods is greater than a hardness of the auxiliary support rod.

Furthermore, the main orthopedic band, the abdominal band and the two shoulder straps together constitute an adjustable encircling support framework.

Furthermore, an overall tightness of the adjustable encircling support framework is cooperatively adjusted by the connection length between the two pulling straps and the abdominal band as well as a tightening degree of the waist compression strap.

Furthermore, when the two pulling straps are under a traction force, the two rib limiting straps are configured to convert a portion of the traction force into a gentle wrapping and limiting force applied to a rib region of the user

The beneficial effects of the present disclosure are as follows:

Through the arrangement of the above-mentioned structure, during use:

On one hand, effective transmission and conversion of forces are achieved: through the force transmission path of “shoulder strap—pulling strap—limiting portion”, the longitudinal pulling force under the armpit is converted into transverse tightening force on the back, which constitutes the fundamental mechanical basis for opening the shoulders and correcting kyphosis.

On the other hand, a stable front-and-back clamping structure is formed: the main orthopedic band provides posterior support, while the abdominal band and waist compression strap provide anterior restraint. Together they form a stable encircling force loop around the torso, fundamentally solving the long-standing problem of conventional posture correction devices being prone to upward displacement, thereby ensuring continuous and stable application of corrective forces.

At the same time, a main supporting framework is provided: the integrated back-lumbar main orthopedic band, together with the built-in main support rod, constitutes the primary structural framework of the entire posture correction device and serves as the foundation for realizing spinal column support.

### BRIEF DESCRIPTION OF DRAWINGS

To more clearly illustrate the technical solutions in the embodiments of the present disclosure, a brief introduction is given below to the accompanying drawings required for describing the embodiments. The drawings described below are only some embodiments of the present disclosure. For those of ordinary skill in the art, other drawings may be obtained based on these drawings without any creative effort. It should be noted that the drawings are not drawn to a 1:1 scale, and the relative sizes of various components are merely illustrated schematically in the drawings and are not necessarily drawn in true proportion. The present disclosure will be further described below in conjunction with the accompanying drawings and specific embodiments.

FIG. 1 is a schematic structural diagram of the posture correction device according to an embodiment of the present disclosure in a wearing state from a front perspective.

FIG. 2 is a schematic structural diagram of the posture correction device in the wearing state from a rear perspective.

FIG. 3 is a schematic structural diagram of the posture correction device in the wearing state from a side perspective.

FIG. 4 is a schematic structural diagram of an arc-shaped support portion of the posture correction device.

FIG. 5 is a schematic structural diagram of an auxiliary support rod of the posture correction device.

FIG. 6 is an exploded view of a partial structure of the posture correction device of the present disclosure in the wearing state.

FIG. 7 is a schematic structural diagram of the posture correction device of the present disclosure in a flat laid-out state.

### DESCRIPTION OF THE REFERENCE NUMERAL

1 main orthopedic band, 2 main support component, 3 auxiliary support component, 4 main support rod, 5 abdominal band, 6 shaping support rod, 7 rib limiting strap, 8 shoulder strap, 9 pulling strap, 10 limiting portion, 10a first sliding groove, 10b second sliding groove, 11 waist compression strap, 11a upper compression strap, 11b lower compression strap, 12 arc-shaped support portion, 13 auxiliary support rod.

### DETAILED DESCRIPTION OF THE EMBODIMENTS

To make the above-mentioned objects, features and advantages of the present disclosure more apparent and readily understandable, specific embodiments of the present disclosure will be described in detail below with reference to the accompanying drawings.

In the following description, numerous specific details are set forth in order to provide a thorough understanding of the present disclosure. However, the present disclosure may be embodied in many other ways different from those described herein, and persons skilled in the art may make similar modifications without departing from the spirit and scope of the present disclosure. Therefore, the present disclosure is not to be limited to the specific embodiments disclosed below.

In the description of the present disclosure, it is to be understood that, when terms such as “center,” “longitudinal,” “lateral,” “length,” “width,” “thickness,” “upper,” “lower,” “front,” “rear,” “left,” “right,” “vertical,” “horizontal,” “top,” “bottom,” “inner,” “outer,” “clockwise,” “counterclockwise,” “axial,” “radial,” “circumferential” and the like appear, these terms indicate orientations or positional relationships based on the orientations or positional relationships shown in the drawings, and are merely for convenience of describing the present disclosure and simplifying the description, and are not intended to indicate or imply that the referred device or element must have a particular orientation, be constructed and operated in a particular orientation, and thus should not be construed as limiting the present disclosure.

Furthermore, when terms such as “first” and “second” appear, they are used merely for descriptive purposes and are not to be construed as indicating or implying relative importance or implicitly specifying the number of indicated technical features. Thus, a feature defined as “first” or “second” may explicitly or implicitly include at least one such feature. In the description of the present disclosure, unless otherwise expressly and specifically defined, the term “plurality” or “a plurality of” means at least two, for example two, three, etc.

In the present disclosure, unless otherwise expressly specified and defined, terms such as “mounted,” “connected,” “linked,” “fixed” and the like shall be understood in a broad sense. For example, it may be a fixed connection, a detachable connection, or an integral connection; it may be a mechanical connection or an electrical connection; it may be directly connected or indirectly connected through an intermediate medium, may be internal communication between two elements or interaction relationship between two elements, unless expressly defined otherwise. For those of ordinary skill in the art, the specific meanings of the above terms in the present disclosure can be understood according to specific circumstances.

In the present disclosure, unless otherwise expressly specified and defined, when a first feature is described as being “on” or “under” a second feature or similar descriptions, it may mean that the first and second features are in direct contact, or that the first and second features are in indirect contact through an intermediate medium. Moreover, that the first feature is “above,” “over,” or “on top of” the second feature may mean that the first feature is directly above or obliquely above the second feature, or merely that the horizontal height of the first feature is higher than that of the second feature. That the first feature is “below,” “under,” or “beneath” the second feature may mean that the first feature is directly below or obliquely below the second feature, or merely that the horizontal height of the first feature is lower than that of the second feature.

It should be noted that when an element is referred to as being “fixed to” or “disposed on” another element, it may be directly on the other element or there may be an intervening element present. When an element is referred to as being “connected to” another element, it may be directly connected to the other element or there may simultaneously be an intervening element. The terms “vertical,” “horizontal,” “upper,” “lower,” “left,” “right,” and similar expressions used in the present disclosure are for illustrative purposes only and are not intended to indicate the only possible implementation.

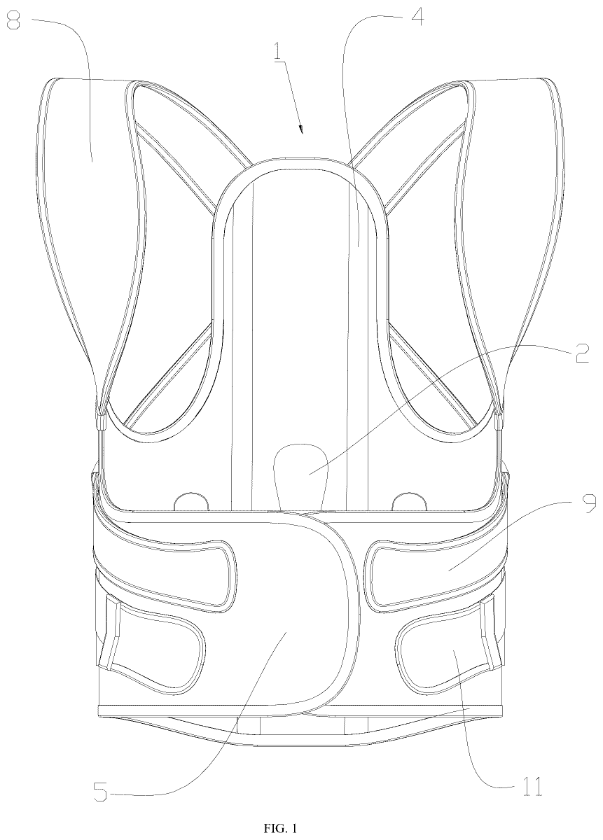

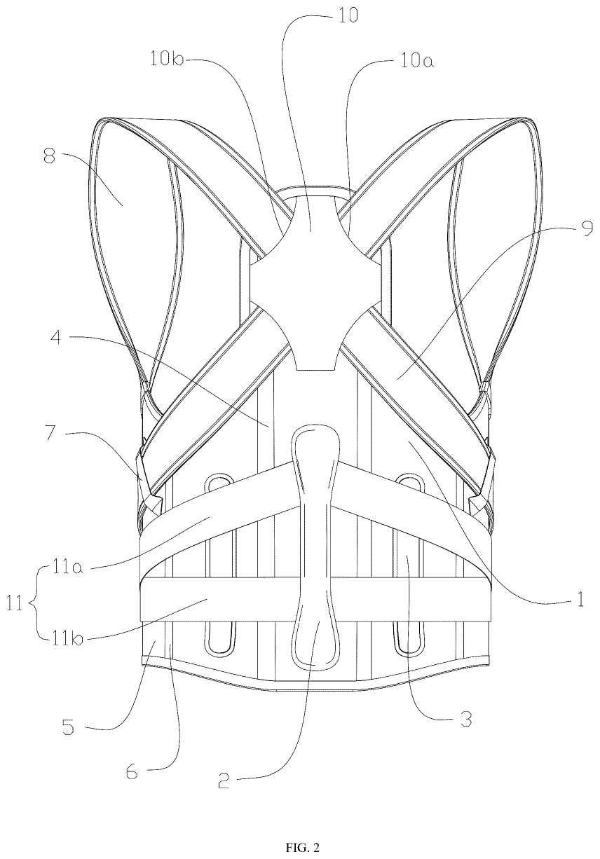

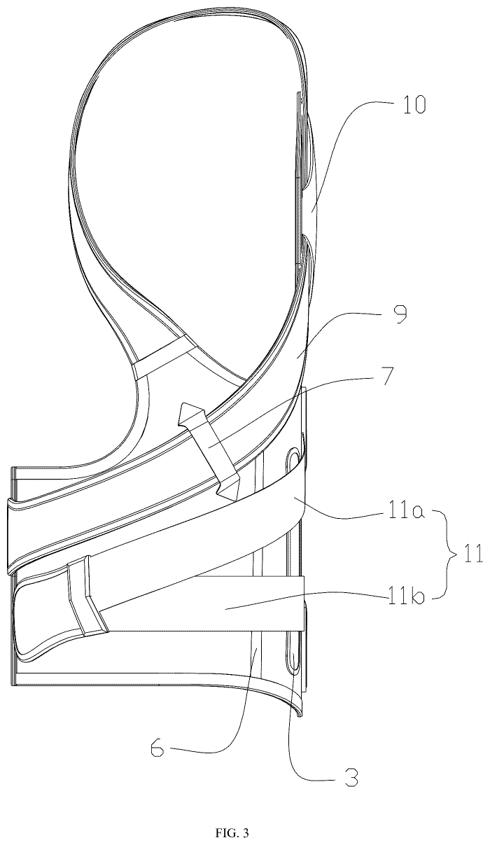

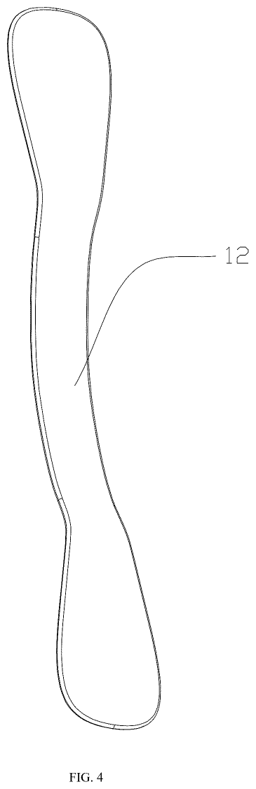

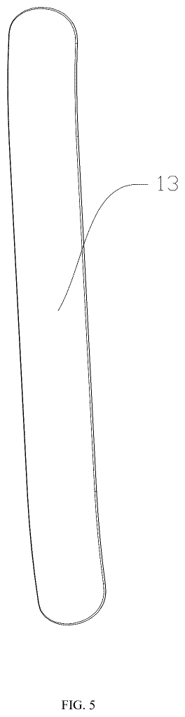

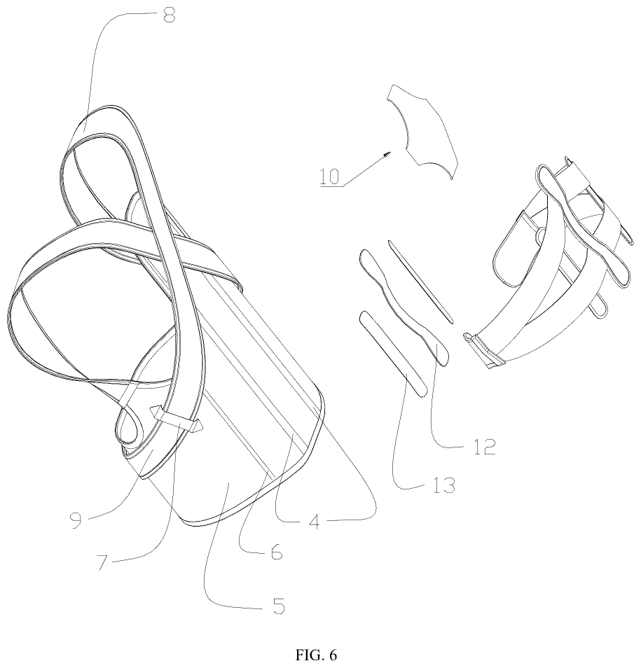

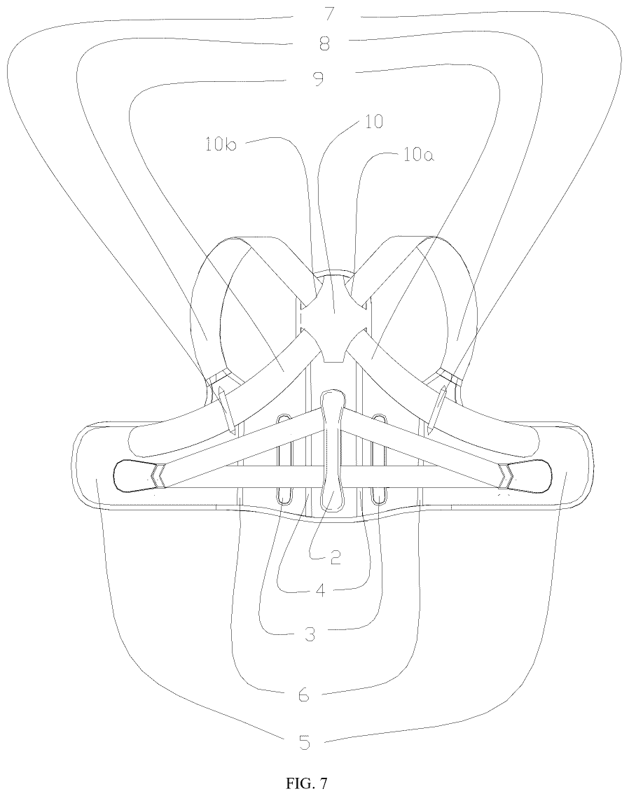

Referring to FIGS. 1-7, there is provided a posture correction device, including: a posture correction device, including a main orthopedic band 1, an abdominal band 5, two shoulder straps 8 and two pulling straps 9. And at least two main support rods 4 are embedded in the main orthopedic band 1 along a spinal direction, and a main support component 2 is provided in the middle part of the main orthopedic band 1.

The abdominal band 5 is symmetrically arranged at two lower lateral sides of the main orthopedic band 1, and the main orthopedic band 1 is fixed to the waist of the user through a free end of the abdominal band 5, the main orthopedic band 1 and the free end of the abdominal band 5 are adjustably and fixedly connected.

The two shoulder straps 8 are symmetrically arranged at upper lateral sides of the main orthopedic band 1, and are respectively configured to extend from under the left and right armpits of the user, pass over the shoulders, and reach the back;

Each of the two pulling straps 9 is fixedly connected to one of the two shoulder straps 8, and each of the two shoulder straps 8 is secured to the respective shoulder of the user through a free end of a corresponding pulling strap 9.

By adjusting a connection length between the two pulling straps 9 and the abdominal band 5, a traction system constituted by the two shoulder straps 8 and the two pulling straps 9 cooperates with a support system constituted by the main orthopedic band 1, thereby achieving posture correction and stable support of the shoulder-back region and the waist-abdominal region.

With the above structural arrangement, in use, the user only needs to perform the core operation of adjusting the connection length between the two pulling straps 9 and the abdominal band 5, which can trigger a systematic mechanical adjustment process: the traction system formed by the two shoulder straps 8 and pulling straps 9 generates backward pulling force→the support system formed by the main orthopedic band 1 provides longitudinal stability→the two systems act synergistically on the shoulder-back and waist-abdominal regions of the user.

This effectively addresses the drawbacks of conventional posture correction devices, such as single-direction corrective force and the need for independent adjustment at multiple body parts, and realizes the advantageous effect of “single adjustment, whole-body coordinated linkage”, which is particularly suitable for daily office work, study and other scenarios requiring prolonged sitting posture.

By providing an adjustable connection between the terminal end of the traction system (two pulling straps 9) and the waist-abdominal encircling system (abdominal band 5), the backward pulling force in the scapular region is skillfully partially converted into horizontal wrapping force and upward lifting force applied to the waist-abdominal region. This force coupling and redistribution mechanism enables the corrective force to be evenly and reasonably distributed throughout the middle and upper trunk, avoiding localized pressure and discomfort caused by force concentration in the armpit or shoulder areas.

Meanwhile, the at least two main support rods 4 embedded along the spinal direction, together with the main support component 2, jointly form a longitudinal support structure combining rigidity and flexibility, which effectively restricts excessive spinal curvature, maintains physiological curvature, while still allowing natural movement of the wearer in the correct posture, thereby improving structural reliability and wearing compliance.

The scientifically designed path of the two shoulder straps 8 passing from under the armpits over the shoulders, combined with the adjustable fixation at all key connection points (shoulders and waist), ensures that the product can closely fit users of different body shapes and enables personalized fine adjustment of tightness. This significantly reduces the risks commonly associated with conventional products, such as slippage, indentation marks, or impaired local circulation, thereby improving long-term wearing comfort and user compliance, and thus facilitating consolidation of long-term correction effects. The adjustment logic of the overall structure is intuitive and clear, greatly reducing the learning cost for correct use by the user.

On this basis, the synergistic system further improves the precision and safety of correction. By adjusting the length of the two pulling straps 9, the user can precisely control the magnitude of traction force applied to the shoulder-back region, realizing multi-level adjustment ranging from gentle reminding to strong correction, thereby meeting the needs of different correction stages; while the stable support provided by the main orthopedic band 1 ensures the correct direction of the corrective force, preventing secondary spinal injury caused by improper external force. The intensity of correction and the stability of support cooperate with each other, enabling the posture correction device to achieve the goal of efficient correction while fully ensuring operational safety.

The overall structure, through the organic linkage of the traction system and the support system as well as intelligent force transmission, constructs a three-dimensional correction network with unified direction and reasonable force distribution. While pursuing high-efficiency correction functionality, the design also fully considers ergonomic comfort and operational convenience, making scientific posture management an easily integrable daily health habit. It should be noted that the main orthopedic band 1 preferably adopts a sandwich structure, the outer layers are breathable mesh fabric, the middle layer is a slow-resilience cushioning material layer, and the outermost layer is elastic fabric or nylon blended fabric.

In this embodiment, the posture correction device further includes a waist compression strap 11. The waist compression strap 11 is fixedly connected to both sides of the main support component 2, and connects the main support component 2 at the back with the abdominal band 5 at both sides by a free end of waist compression strap 11. The free end of waist compression strap 11 is adjustably and fixedly connected to the abdominal band 5. By virtue of the above structural arrangement, during use, the waist compression strap 11 establishes an adjustable connection between the main support component 2 at the back and the abdominal band 5 at both sides, thereby integrally coupling the originally relatively independent back support system and the waist-abdomen encircling system into a framework structure exhibiting significantly enhanced overall integrity. This configuration markedly improves the structural stability of the entire posture correction device and the efficiency of force transmission. At the same time, by adjusting the tightness of the waist compression strap 11, the user can independently control the lateral and oblique wrapping pressure applied to both sides of the waist and the lower back region. Such adjustability enables synergistic cooperation with the traction force of the two shoulder straps and the binding force of the abdominal band, thereby achieving fine-grained and personalized adjustment of the support strength and wrapping configuration in the lumbar-abdominal region, so as to more precisely accommodate the correction requirements of users having different body types.

Further, the transverse tightening action provided by the waist compression strap 11 cooperates with the main support component 2 to more securely conform and position the lumbar vertebral region of the user against a preset physiological curvature support. This arrangement effectively enhances the constraint and correction capability with respect to adverse postures such as anterior pelvic tilt and excessive lumbar lordosis, thereby improving the stability of overall posture maintenance and the correction efficacy.

In this embodiment, the posture correction device further includes auxiliary support components 3 symmetrically arranged on both sides of the main support component 2. By adjusting the connection length between the two pulling straps 9 and the abdominal band 5 and tightening the waist compression strap 11, the traction system constituted by the two shoulder straps 8 and the two pulling straps 9, the support system constituted by the main orthopedic band 1, and the encircling system constituted by the abdominal band 5 act in coordination, thereby achieving posture correction and stable support for the shoulder-back and waist-abdominal regions.

By virtue of the above structural arrangement, during use, the auxiliary support components 3 symmetrically disposed on both sides of the main support component 2 effectively enlarge the coverage area of rigid support at the back. This enables more uniform distribution of corrective forces to the muscle groups and soft tissues on both sides of the lumbar vertebrae, avoiding excessive local pressure and significantly improving wearing comfort. Moreover, the introduction of the auxiliary support components 3 adds a lateral wing stabilization system in addition to the two shoulder straps traction system, the main support system, and the abdominal band encircling system. By synchronously adjusting the two pulling straps 9 (controlling traction) and the waist compression strap 11 (controlling lateral wrapping), the user can achieve dynamic balancing and precise distribution of corrective forces across the four regions of shoulders, back, waist, and abdomen, thereby forming a three-dimensional, multi-level coordinated correction mechanism.

In this embodiment, the main support component 2 has embedded therein an arc-shaped support portion 12 adapted to the physiological curvature of the lumbar vertebrae, and the auxiliary support components 3 symmetrically arranged on both sides of the main support component 2 are provided with an auxiliary support rod 13. By virtue of the above structural arrangement, during use, the arc-shaped support portion 12 embedded in the main support component 2 is specifically designed with a curvature that matches the natural physiological lordosis of the human lumbar spine. It can precisely conform to the lumbar vertebral region, providing stable and directional upward supporting force, effectively maintaining the normal lordotic curvature of the lumbar vertebrae, thereby scientifically correcting adverse postures such as anterior pelvic tilt and lumbar kyphosis while avoiding inappropriate compression on the spinal column. As a core rigid component, the arc-shaped support portion 12 significantly enhances the resistance of the main support component 2 to bending in the sagittal plane (anteroposterior direction), thereby strongly restricting hunchback posture. The auxiliary support rod 13 enhances the lateral stability of a corresponding auxiliary support component 3 in the coronal plane (left-right direction), cooperatively restricting lateral curvature of the spine. The combination of both improves the overall structural performance of the posture correction device in counteracting complex adverse postures.

In this embodiment, the back of the main orthopedic band 1 is provided with a limiting portion 10, and each of the two pulling straps 9 passes through the limiting portion 10 in an X-shaped crossing manner so as to restrict a stretching direction of the two pulling straps 9. By virtue of the above structural arrangement, during use, the limiting portion 10, through its X-shaped crossing structure, physically constrains the stretching paths of the two pulling straps 9, ensuring that the traction forces generated thereby are precisely directed obliquely downward. This enables the traction forces to more effectively act on the medial aspect of the scapulae and the middle-to-lower back muscle groups, promoting scapular adduction and depression, and significantly improving the correction accuracy for rounded shoulder and hunchback postures. At the same time, the X-shaped crossing design mechanically couples the left and right pulling straps 9 in the central region of the back, forming a stable “force anchor point” that effectively prevents lateral slippage of the two pulling straps 9 under load, thereby ensuring efficient and stable transmission of traction forces along the preset paths to the waist-abdomen encircling system, and enhancing the structural rigidity and motion consistency of the entire posture correction device system.

In this embodiment, the abdominal band 5 is provided with two rib limiting straps 7. Each of the two pulling straps 9 sequentially passes through the limiting portion 10 and a respective rib limiting strap of the two rib limiting straps 7, and ultimately secures the two shoulder straps 8 to the user's shoulder by the free ends of the two pulling straps 9, that is adjustably and fixedly connected. By virtue of the above structural arrangement, during use, the two rib limiting straps 7 serves as an intermediate guide and force application point located on the lateral side of the body, altering the purely longitudinal traction path of the two pulling straps 9. When the two pulling straps 9 are under tension, a portion of the upward lifting force is converted, via the two rib limiting straps 7, into a gentle circumferential wrapping force and lateral stabilizing force applied to the lower margin of the rib cage. This more effectively gathers and stabilizes the position of the ribs, assists in improving rib flaring caused by improper breathing patterns or posture, and enriches the hierarchy and precision of force application in the correction process. The two rib limiting straps 7 physically connect and mechanically couples the traction system (two shoulder straps 8/two pulling straps 9) with the encircling system (abdominal band 5) on the lateral aspect of the human body, such that the front and rear systems are no longer independent modules. This enhances the overall attachment feel and stability of the posture correction device on the user's torso, reduces possible relative displacement or loosening of components during movement, and ensures sustained and stable correction efficacy.

In this embodiment, the limiting portion 10 includes a first sliding groove 10a and a second sliding groove 10b crossing each other in an X shape, and the two pulling straps 9 are slidably threaded along the first sliding groove 10a and the second sliding groove 10b, respectively. By virtue of the above structural arrangement, during use, the sliding groove structure continuously maintains the preset X-shaped crossing spatial relationship between the two pulling straps 9 throughout user movement, preventing mutual entanglement or deviation from the correct path due to body motion. This ensures that the traction system of the posture correction device can continuously and reliably provide corrective forces meeting the design requirements under various dynamic postures, thereby improving the dynamic stability of the product.

In this embodiment, the waist compression strap 11 includes an upper compression strap 11a and a lower compression strap 11b. The upper compression strap 11a is fixedly connected to an upper lateral position of the main support component 2, and the lower compression strap 11b is fixedly connected to a lower lateral position of the main support component 2. By virtue of the above structural arrangement, during use, the upper compression strap 11a and the lower compression strap 11b form two connection anchor points at different heights with the main support component 2, and are then jointly connected to the abdominal band 5. This design constructs an approximately trapezoidal stable force transmission framework between the back and the lateral waist, greatly enhancing the overall connection strength and torsional resistance of the posterior and lateral structures of the posture correction device, and effectively preventing displacement or loosening of the support structure during movement.

In this embodiment, after the free ends of the upper compression strap 11a and the lower compression strap 11b are fixedly connected to each other, the main support component 2 at the back is connected to the abdominal band 5. By virtue of the above structural arrangement, during use, this connection manner causes the upper compression strap 11a and the lower compression strap 11b to integrate into a single mechanical entity in the lateral waist region, thereby forming, together with the abdominal band 5, a complete closed pressurizing loop. This ensures that the pressures applied to the upper and lower segments of the waist can be interlinked and uniformly transmitted, thereby achieving coordinated control of stable support for the upper lumbar segment and pelvic constraint for the lower lumbar segment, and improving the overall integrity of lumbar correction.

In this embodiment, the abdominal band 5 employs a first hook-and-loop fastener to achieve an adjustable fixed connection. The hook surface of the first hook-and-loop fastener is disposed on the inner side of the abdominal band 5, and the loop surface is disposed on the outer side of the abdominal band 5. With the above structural arrangement, during use, the hook surface is positioned on the inner side of the abdominal band facing the body, such that after wearing, the hook surface is completely covered by the abdominal band itself, while only the soft loop surface is exposed on the outer side. This configuration effectively prevents the rigid hook surface from accidentally catching on or abrading outer clothing during daily activities, thereby significantly enhancing the product's protectiveness toward worn garments and improving its universal applicability. When fastening, the user can directly cover and press the outer loop surface onto the inner hook surface in an intuitive and rapid operation. It should be noted that the hook surface is provided only on the inner side of one side of the abdominal band 5, while the loop surface is provided on the outer side of both sides of the abdominal band 5.

In this embodiment, each of the two pulling straps 9 employs a second hook-and-loop fastener to achieve an adjustable fixed connection, the hook surface of the second hook-and-loop fastener is disposed on the pulling strap 9. With the above structural arrangement, during use, the user can precisely and continuously control the effective working length of the pulling strap 9 by freely adjusting the bonding position of the second hook-and-loop fastener on the strap body, thereby achieving stepless fine adjustment of the magnitude of traction force applied to the shoulder and back regions. This design enables millimeter-level micro-adjustment of the traction force according to the user's real-time physical sensation and correction requirements, while the reliable locking provided by the second hook-and-loop fastener ensures that the preset traction force remains stable and does not become accidentally loosened or ineffective during activities.

In this embodiment, the waist compression strap 11 employs a third hook-and-loop fastener to achieve an adjustable fixed connection, the hook surface of the third hook-and-loop fastener is disposed on the waist compression strap 11. With the above structural arrangement, during use, the user can continuously and precisely control the degree of circumferential tightening applied to the lateral waist and abdominal regions by freely adjusting the bonding position of the waist compression strap 11. This enables millimeter-level micro-adjustment of both the transverse wrapping force and the oblique lifting force on the waist, while the reliable locking of the third hook-and-loop fastener ensures that the preset pressure remains stable during body movements, preventing loosening. By integrating the hook surface of the third hook-and-loop fastener directly onto the compression strap itself, the need for exposed rigid buckles or excessively long strap tails on the lateral waist is eliminated, resulting in a cleaner and more streamlined appearance in the lateral waist region of the product, while effectively reducing the possibility of hooking or friction with external clothing.

In this embodiment, the main orthopedic band 1 is configured as an integrated back-waist structure. With the above structural arrangement, during use, the integrated structure eliminates physical discontinuities or connecting seams between the back support region and the waist support region, enabling the at least two main support rods 4 and the main support component 2 to form a continuous and uninterrupted mechanical support path along the spine. This ensures uniform and continuous transmission of corrective force along the spine from top to bottom, effectively avoiding stress concentration, support force attenuation, or structural wobbling that may occur at connection points in conventional split-type designs, thereby providing a more stable and robust support foundation for the entire trunk.

In this embodiment, the two main support rods 4 are symmetrically arranged parallel to the centerline of the spine. With the above structural arrangement, during use, the symmetrical and parallel arrangement enables the at least two main support rods 4 to be precisely positioned on both sides of the spine, forming symmetrical rigid support bars on either side of the body's midline. This provides balanced bilateral clamping force to the spine, effectively restricting abnormal lateral curvature tendencies of the spine in the coronal plane (left-right direction), and assisting in maintaining symmetry of left-right trunk posture.

In this embodiment, a shaping support rod 6 is embedded in a connection portion between the abdominal band 5 and the main orthopedic band 1. With the above structural arrangement, during use, the connection portion serves as the critical hub where abdominal circumferential force and longitudinal force of the main support system intersect and transmit, and is therefore subjected to complex loading. The embedding of the shaping support rod 6 in this region significantly enhances local stiffness and shape retention capability, effectively preventing distortion, collapse, or permanent deformation of the connection portion due to fabric stretching or seam stress during daily wearing, body movement, or repeated adjustments, thereby ensuring long-term reliability of the core structure.

In this embodiment, in the worn state, the main support component 2 and the arc-shaped support portion 12 are positioned and attached to the user's lumbar vertebral region, while the auxiliary support component 3 and the auxiliary support rod 13 are positioned and attached to the bilateral regions of the lumbar vertebrae. The hardness of the arc-shaped support portion 12 is greater than that of the auxiliary support rod 13. With the above structural arrangement, during use, through precise regional positioning (central lumbar vertebrae corresponding to the core, bilateral regions corresponding to muscle groups) combined with differentiated hardness configuration, the design establishes a clear primary-secondary, target-specific three-dimensional support structure. This not only ensures that corrective forces are accurately applied to target biomechanical units (such as vertebral bodies and deep muscle groups), but also makes the mechanical behavior of the entire support system more predictable and controllable, thereby improving the effectiveness and safety of correction.

In this embodiment, a hardness of the arc-shaped support portion 12 is greater than that of the at least two main support rods 4, and a hardness of the at least two main support rods 4 is greater than that of the auxiliary support rod 13. With the above structural arrangement, during use, the three-level gradient hardness configuration achieves more synergistic mechanical coupling among the traction system, core support system, and lateral stabilization system. During dynamic activities, the stable support of the core region provides a reliable “force benchmark” for the body, while the moderately flexible lateral support allows reasonable lateral flexion and rotation of the trunk within safe limits, thereby balancing correction effectiveness with flexibility for daily activities, making the product more suitable for prolonged wearing and dynamic use.

In this embodiment, the main orthopedic band 1, the abdominal band 5, and the two shoulder straps 8 together constitute an adjustable encircling support framework. With the above structural arrangement, during use, the encircling framework organically integrates the originally independent functional modules of back support, waist wrapping, and shoulder traction into a unified mechanical whole. When the user performs adjustment, the components within the framework produce linked responses, forming a synergistic “one-pull-multi-tightening” effect, enabling corrective forces to act evenly and synchronously on the anterior, posterior, and lateral aspects of the trunk, thereby significantly enhancing the systematic nature and overall efficiency of posture correction.

In this embodiment, an overall tightness of the adjustable encircling support framework is cooperatively adjusted through the connection length between the pulling strap 9 and the abdominal band 5, as well as the fastening degree of the waist compression strap 11. With the above structural arrangement, during use, by linked adjustment of the length of the pulling strap 9 (controlling shoulder-back traction force) and the tightness of the waist compression strap 11 (controlling lateral waist-abdominal wrapping force), the user can synchronously change the overall tightening degree and internal tension distribution of the adjustable encircling support framework in a one-step manner. This ensures that traction force, lateral pressure, and core support force always remain in dynamic equilibrium, avoiding incoordination problems where one region is excessively tightened while others become loose, thereby achieving overall optimization of corrective forces.

In this embodiment, when the two pulling straps 9 are under a traction force, the two rib limiting straps 7 are configured to convert a portion of the traction force into gentle wrapping and limiting force applied to a rib region of the user. With the above structural arrangement, during use, the traction force is precisely transformed, through the guidance of the two rib limiting straps 7, into gentle circumferential wrapping force on the lower margin of the rib cage, thereby achieving accurate lateral positioning and stabilization of the ribs and helping to improve rib flaring caused by poor posture. At the same time, this design modifies the purely longitudinal traction path of the two pulling straps 9, converting part of the upward lifting force into lateral stabilizing force on the ribs, thereby enriching the hierarchy of corrective force application and making the mechanical intervention more three-dimensional and efficient.

It should be noted that the two rib limiting straps 7 and the limiting buckle 10 are made of the same material, namely microfiber leather, and the material covering and fixing the arc-shaped support portion 12 on the exterior of the main support component 2 is the same as the material covering and fixing the auxiliary support rod 13 on the exterior of the auxiliary support component 3, both being ultra-soft polyurethane (PU) leather.

The foregoing describes one or more embodiments provided in conjunction with specific content, and it should not be construed that the specific implementations of the present disclosure are limited only to these descriptions. Any methods, structures, or the like that are approximate or identical to those of the present disclosure, or any technical deductions or substitutions made based on the inventive concept of the present disclosure, shall be deemed to fall within the protection scope of the present disclosure.

What is claimed is

1. A posture correction device, comprising:
a main orthopedic band, wherein at least two main support rods are embedded in the main orthopedic band along a spinal direction, and a main support component is provided in a middle part of the main orthopedic band;
an abdominal band symmetrically arranged at two lower lateral sides of the main orthopedic band, wherein the main orthopedic band is configured to be fixed to a waist of a user through a free end of the abdominal band;
two shoulder straps symmetrically arranged at upper lateral sides of the main orthopedic band, each of the two shoulder straps is configured to extend from under left and right armpits of the user, pass over a respective shoulder, and reach a back of the user;
two pulling straps, wherein each of the two pulling straps is fixedly connected to one of the two shoulder straps, and each of the two shoulder straps is secured to the respective shoulder;
wherein by adjusting a connection length between the two pulling straps and the abdominal band, a traction system constituted by the two shoulder straps and the two pulling straps cooperates with a support system constituted by the main orthopedic band to achieve posture correction and stable support of a shoulder-back region and a waist-abdominal region of the user.

2. The posture correction device according to claim 1, wherein the posture correction device further comprises a waist compression strap fixedly connected to both sides of the main support component, and the main support component is connected with the abdominal band by a free end of the waist compression strap.

3. The posture correction device according to claim 2, wherein the posture correction device further comprises auxiliary support components, wherein the auxiliary support components are symmetrically arranged on two sides of the main support component;
wherein by adjusting the connection length between the two pulling straps and the abdominal band and tightening the waist compression strap, the traction system constituted by the two shoulder straps and the two pulling straps, the support system constituted by the main orthopedic band, and an encircling system constituted by the abdominal band cooperate to achieve posture correction and stable support of the shoulder-back region and the waist-abdominal region.

4. The posture correction device according to claim 3, wherein an arc-shaped support portion adapted to a physiological curvature of a lumbar spine is embedded in the main support component, and an auxiliary support rod is provided inside each of the auxiliary support components.

5. The posture correction device according to claim 1, wherein a limiting portion is provided on the main orthopedic band, and each of the two pulling straps is passed through the limiting portion in an X-shaped crossing manner, so as to restrict a stretching direction of the two pulling straps.

6. The posture correction device according to claim 5, wherein two rib limiting straps are provided on the abdominal band, and each of the two pulling straps sequentially passes through the limiting portion and a respective rib limiting strap of the two rib limiting straps, and the respective rib limiting strap of the two rib limiting straps is finally configured to fix one of the two shoulder straps to the respective shoulder of the user by the free end of a respective pulling strap of the two pulling straps.

7. The posture correction device according to claim 5, wherein the limiting portion comprises a first sliding groove and a second sliding groove crossing each other in an X shape, and the two pulling straps are slidably threaded along the first sliding groove and the second sliding groove, respectively.

8. The posture correction device according to claim 2, wherein the waist compression strap comprises an upper compression strap and a lower compression strap, wherein the upper compression strap is fixedly connected to an upper lateral side of the main support component, and the lower compression strap is fixedly connected to a lower lateral side of the main support component.

9. The posture correction device according to claim 8, wherein after free ends of the upper compression strap and the lower compression strap are fixedly connected to the main support component, the main support component is connected with the abdominal band.

10. The posture correction device according to claim 1, wherein the abdominal band is a first hook-and-loop fastener, wherein a hook surface of the first hook-and-loop fastener is disposed on an inner side of the abdominal band, and a loop surface of the first hook-and-loop fastener is disposed on an outer side of the abdominal band.

11. The posture correction device according to claim 1, wherein each of the two pulling straps is a second hook-and-loop fastener, wherein a hook surface of the second hook-and-loop fastener is disposed on each of the two pulling straps.

12. The posture correction device according to claim 2, wherein the waist compression strap is a third hook-and-loop fastener, wherein a hook surface of the third hook-and-loop fastener is disposed on the waist compression strap.

13. The posture correction device according to claim 1, wherein the main orthopedic band is of an integrated back-waist structure.

14. The posture correction device according to claim 1, wherein the at least two main support rods are symmetrically arranged parallel to a spine centerline.

15. The posture correction device according to claim 1, wherein a shaping support rod is embedded in a connection portion between the abdominal band and the main orthopedic band.

16. The posture correction device according to claim 4, wherein, when the posture correction device is in a worn state, the main support component and the arc-shaped support portion are positioned to fit a lumbar region of the user, the auxiliary support components and the auxiliary support rod are positioned to fit regions on both sides of the lumbar spine, and a hardness of the arc-shaped support portion is greater than a hardness of the auxiliary support rod.

17. The posture correction device according to claim 4, wherein a hardness of the arc-shaped support portion is greater than a hardness of the at least two main support rods, and the hardness of the at least two main support rods is greater than a hardness of the auxiliary support rod.

18. The posture correction device according to claim 2, wherein the main orthopedic band, the abdominal band and the two shoulder straps together constitute an adjustable encircling support framework.

19. The posture correction device according to claim 18, wherein an overall tightness of the adjustable encircling support framework is cooperatively adjusted by the connection length between the two pulling straps and the abdominal band as well as a tightening degree of the waist compression strap.

20. The posture correction device according to claim 6, wherein, when the two pulling straps are under a traction force, the two rib limiting straps are configured to convert a portion of the traction force into a gentle wrapping and limiting force applied to a rib region of the user.
